(19) United States
(12) Patent Application Publication
(10) Pub. No.: US 2008/0243010 A1
(43) Pub. Date: Oct. 2, 2008

(54) SYSTEM AND METHOD FOR FACILITATING PROPER CUFF USE DURING NON-INVASIVE BLOOD PRESSURE MEASUREMENT

(75) Inventor: Robert Stanley Kulik, Tampa, FL (US)

Correspondence Address: PETER VOGEL;GE HEALTHCARE, 20225 WATER TOWER BLVD., MAIL STOP W492, BROOKFIELD, WI, 53045, US

(73) Assignee: GENERAL ELECTRIC COMPANY, Schenectady, NY (US)

(21) Appl. No.: 11/694,585

(22) Filed: Mar. 30, 2007

(51) Int. Cl. A61B 5/022

(52) U.S. Cl. 600/499

(57) ABSTRACT

A non-invasive blood pressure system comprises a blood pressure cuff having an identifier and an aperture such that the identifier can be read through the aperture when the cuff is in a desired configuration. Another comprises a processor in operative communication with the cuff and configured to initiate a blood pressure determination when the identifier can be read through the aperture, while a method provides such blood pressure cuffs.

### FIELD OF INVENTION

In general, the inventive arrangements relate to non-invasive blood pressure NIBP) monitoring. More particularly, they relate to facilitating correct sizing and usage of blood pressure cuffs during NIBP measurements.

### BACKGROUND OF INVENTION

Determining patient blood pressure is a common occurrence in many hospitals, clinics, physician offices, and/or the like, occurring thousands of times each day around the world. Commonly, automated NIBP systems inflate and deflate pneumatic cuffs that are wrapped around a patient's limb, particularly while measuring an amplitude of pressure fluctuations caused by a patient's pulse. The use of an appropriate blood pressure cuff for a particular patient's limb size is a key element in obtaining an accurate and timely blood pressure determination, as are correct wrapping techniques.

In actual practice, however, clinicians frequently apply the wrong size cuff and/or wrap it incorrectly because of inadequate training in using NIBP systems and/or not specifically being aware of the effects of wrongly-sized cuffs and/or incorrect applications. For example, in many instances, operators may not have a tape measure at hand to determine a patient's proper cuff size, or they may lack a selection of different cuff sizes. Even when these are available, however, the operator may still not select the appropriate cuff for a particular patient.

Another problem that clinicians face while using NIBP systems is the inaccessibility of NIBP measuring devices, particularly, but not limited to, situations where devices are fixed on a wall in some part of a room. While the cuff itself may be attached to a hose of a particular length, and thus readily accessible, the caregiver must still initiate the NIBP determination by actuating a remote button on the system, which may be mounted in an awkward spot, blocked by other equipment and/or furniture, and/or otherwise difficult to reach.

Thus, there exists a need to ensure that during NIBP measurements, the pressure cuff used is appropriately sized for and applied to a particular patient. There is also a need to initiate NIBP determinations remotely, particularly once the cuff is correctly applied.

Accordingly, it would be desirable to provide systems and methods that facilitate using an appropriate cuff during NIBP measurements, wound in a correct manner for a particular patient. It is also desirable to be able to start NIBP determinations remotely upon properly selecting and applying a particular cuff—i.e., without having to interact directly with a NIBP monitor itself.

### SUMMARY OF INVENTION

The above-mentioned shortcomings, disadvantages and problems are addressed herein, and which can be understood by reading and understanding the following specification.

In one embodiment, a non-invasive blood pressure system comprises a blood pressure cuff having an identifier and an aperture such that the identifier can be read through the aperture when the cuff is in a desired configuration.

In another embodiment, a non-invasive blood pressure system comprises the cuff and a processor in operative communication with the cuff and configured to initiate a blood pressure determination when the identifier can be read through the aperture.

And in yet another embodiment, a method provides the blood pressure cuff.

Various other features, objects, and advantages of the inventive arrangements will be made apparent to those skilled in the art from the accompanying drawings and detailed description thereof.

### BRIEF DESCRIPTION OF SEVERAL VIEWS OF THE DRAWINGS

A clear conception of the advantages and features constituting inventive arrangements, and of various construction and operational aspects of typical mechanisms provided by such arrangements, are readily apparent by referring to the following illustrative, exemplary, representative, and non-limiting figures, which form an integral part of this specification, in which like numerals generally designate the same elements in the several views, and in which:

FIG. 1 is a perspective diagram of a blood pressure cuff that visually facilitates the correct usage of an appropriately-sized blood pressure cuff during an NIBP determination;

FIG. 2 is a perspective diagram of an alternative blood pressure cuff of FIG. 1;

FIG. 3 is another perspective diagram of a blood pressure cuff that facilitates the correct usage of an appropriately-sized blood pressure cuff during an NIBP determination;

FIG. 4 is a perspective diagram of an alternative blood pressure cuff of FIG. 3;

FIG. 5 is a block diagram of an NIBP monitoring system according to various embodiments of the inventive arrangements; and

FIG. 6 is a flowchart illustrating a method of facilitating blood pressure determinations using an NIBP monitoring system according to various embodiments of the inventive arrangements.

### DETAILED DESCRIPTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the inventive arrangements.

In an embodiment, a system for collecting NIBP readings from a patient is disclosed. The system uses a mechanism to facilitate the usage of an appropriately-sized blood pressure cuff and appropriate wrapping techniques used in NIBP determinations. The term “appropriate-sized” or “correct cuff size” indicates the cuff pressure size matches the limb size of a patient, particularly according to the specifications provided by the cuff manufacturer and/or the like. For example, a patient may be an infant, child, regular adult, medium-sized adult, or large adult, and/or the like.

In another embodiment, the inventive arrangements provide an automatic NIBP measuring apparatus. The apparatus initializes the NIBP measurement automatically upon confirming the correct usage of the blood pressure cuff.

In another embodiment, the inventive arrangements provide a method for enforcing the usage of correct sized blood pressure cuffs. The method identifies the correct cuff size, determines the validity of the cuff size, and controls a NIBP measurement based on the correct application of the cuff initializing the monitoring.

Even though the described inventive arrangements are explained in reference to NIBP monitoring systems and methods, the inventive concepts may be readily applied to different fields as well, such as packaging and shipping industries, automated mechanical and/or manufacturing industries, and/or the like.

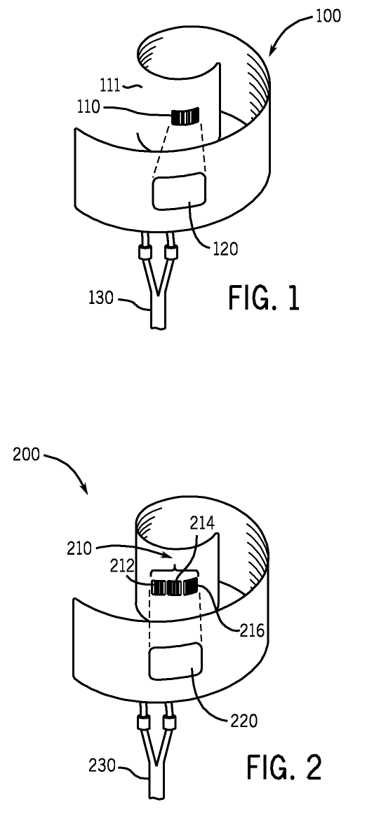

Referring now to FIG. 1, a blood pressure cuff 100 is provided for ensuring the correct use of the cuff 100 during blood pressure measurements. More specifically, the cuff 100 is preferably an inflatable and deflatable cuff arranged to be worn about a patient's limb (not shown) Preferably, the cuff 100 is operatively connected to an NIBP monitor (not shown) used in an NIBP monitoring system (not shown). In use, the cuff 100 is wound around the patient's limb and inflated and deflated during the blood pressure measurement, and a parameter is provided in association with the cuff 100 for identifying the physical and/or operational characteristics of the cuff 100. In one embodiment, the parameter identifies at least the correct size of the cuff 100. For example, in FIG. 1, the cuff 100 is provided with a barcode 110 printed on a surface 111 of the cuff 100 for representing the parameter associated with the cuff 100. Preferably, the barcode 110 may specify at least the size of the cuff 100. The cuff 100 is further provided with an aperture, such as a window 120, for identifying the parameter upon usage of an appropriately-sized cuff and proper wrapping thereof. The window 120 provides a visual opening to assist reading the barcode 110 when the cuff is properly sized and applied to the patient's limb, for example using a barcode reader (not shown). Preferably, the barcode 110 is printed in a location such that it is overlapped when the cuff 100 is wrapped around the patient's limb, but becomes visible through the window 120 when the cuff 100 is properly wrapped around the patient's limb. Accordingly, the positioning of the barcode 110 and window 120 should suitably align when the cuff 100 is wound appropriately around the patient's limb. In addition, the cuff 100 can be further connected to a blood pressure monitor (not shown) through a hose 130 and/or the like.

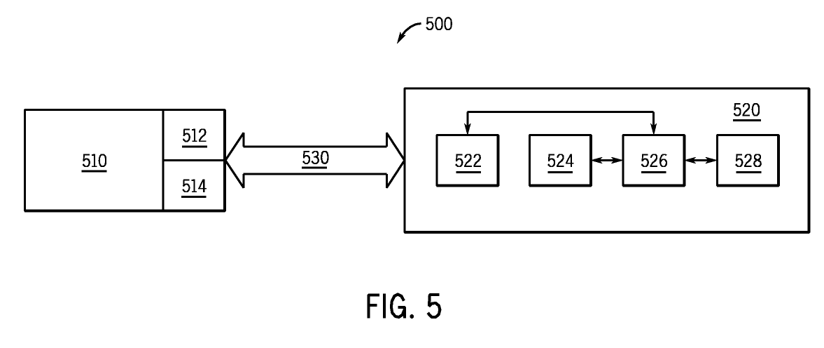

Before initializing a blood pressure measurement, the blood pressure monitor can check for the availability of the barcode 110. For example, if the barcode 110 is visible through the window 120, then the NIBP system can begin determining the patient's blood pressure. In addition, once the barcode 110 is read, it can be communicated to a processor (see FIG. 5) for remote and/or automated controlling of the NIBP system, based on the information read. However, if the barcode 110 is not visible or not read correctly through the window 120, then the NIBP system may not yet begin determining the patient's blood pressure. Instead, the processor may suggest instructions to the caretaker about correct wrapping techniques and/or the like.

In one embodiment, the barcode 110 may be used as locking mechanism, wherein unless the barcode 110 is read correctly through the window 120, the NIBP system may not allow the system and/or an operator to begin determining a patient's blood pressure. In addition, based on the information read from the bar code, the processor may provide additional wrapping instructions to the operator. However, an appropriate override may also be provided to unlock the device and allow it take NIBP measurements even if the barcode 110 was not visible through the window 120 or optimal wrapping was not possible given a particular patient situation.

In one embodiment, the cuff size obtained by reading the barcode 110 may also be compared with pre-determined cuff size information for a specific patient to further ensure the correct size blood pressure cuff is used. For example, if a cuff size interpreted from the barcode 110 does not agree with pre-determined information for a particular patient, the processor may again suggest a correct cuff size and/or provide wrapping instructions to the caretaker. The pre-determined information may be obtained, for example, from a server (not shown) having access to the patient's electronic medical record and/or the like, and/or it may be input to the system by the caregiver.

In an embodiment, the cuff size identified using the barcode 110 can also be sent to a medical record database (not shown) for storing the same, and the medical record database may be accessed using a server by the NIBP monitoring system.

In an embodiment, the processor can also be configured to automatically adjust operational characteristics of the NIBP system based on the read parameters in order to optimize the NIBP readings taken by the respective cuff 100. For example, if the patient is an infant or child, the settings of the monitor can be adjusted accordingly, such as setting an upper limit on the cuff inflation pressure and/or the like.

Referring now to FIG. 2, another blood pressure cuff 200 can also be provided with a plurality of barcodes 210 printed on a surface 211 of the cuff 200 for representing the parameter associated with the cuff 200. For example, the plurality of barcodes 210 may include a first barcode 212, a second barcode 214, and/or a third barcode 216. Preferably, the plurality of bar codes 210 are provided in association with the cuff 200 for identifying the physical and/or operational characteristics of the cuff 200. Again, the cuff 200 is further provided with an aperture, such as a window 220, for identifying the parameter upon usage of an appropriately-sized cuff and/or proper wrapping thereof. The window 220 provides a visual opening to assist reading the plurality of barcodes 210 when the cuff 200 is properly sized and/or applied to the patient's limb, for example using a barcode reader (not shown). Preferably, the plurality of barcodes 210 are printed in a location such that they are overlapped when the cuff 200 is wrapped around the patient's limb, but become visible through the window 220 when the cuff 200 is properly wrapped around the patient's limb. For example, the degree of wrap of the cuff 200 determines which of the plurality of barcodes 210 is visible through the window 220. This, the NIBP monitor knows how tightly the cuff 210 is wrapped around the limb of the patient, and it can use this information to more precisely verify proper cuff application and/or adjust its operation. For example, in the figure, the plurality of barcodes 210 includes the first barcode 212, second barcode 214, and third barcode 216, whereby if first barcode 212 is visible through the window 220, then the cuff 200 may be over-wrapped and the NIBP system may instruct the operator to re-adjust the cuff 200 and/or replace it with one of a different size. Similarly, if the cuff 200 is under-wrapped, then the third barcode 216 may be visible through the window 220 and the NIBP system may again instruct the operator to re-adjust the cuff 200 and/or replace it with one of a different size. And likewise, if the cuff 200 is properly sized and wrapped, then the second barcode 214 may be visible through the window 220 and the NIBP system may begin the blood pressure determination and/or allow the clinician to do so. Once the appropriate-sized cuff is wrapped appropriately around the patient's limb, then the correct barcode of the plurality of barcodes 210 will be visible. And again, the cuff 200 can be further connected to the blood pressure monitor and/or system through a hose 230 and/or the like.

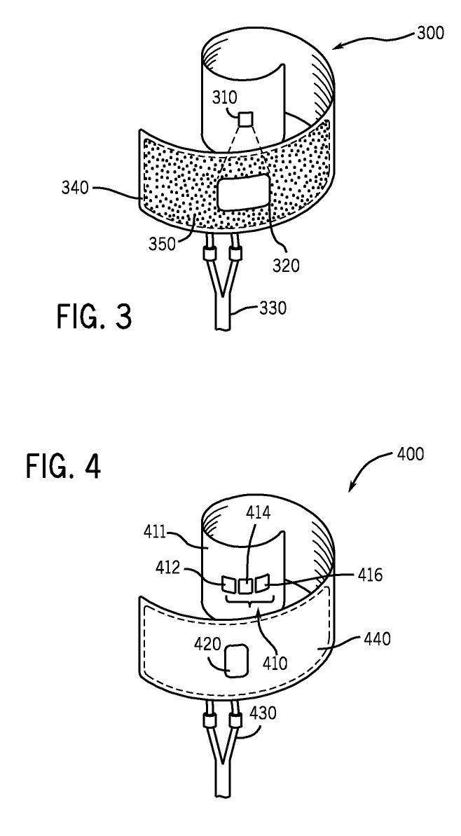

Referring now to FIG. 3, another blood pressure cuff 300 is provided, again with an aperture such as a window 320 and/or a hose 330. More specifically, however, the cuff 300 is wound around the patient's limb and inflated and deflated for monitoring the patient's blood pressure. A parameter is again provided in association with the cuff 300 for identifying the physical and/or operational characteristics of the cuff 300. In an embodiment, the parameter identifies at least the size of the cuff 300. In this embodiment, however, an radio-frequency identification (RFID) tag 310 supplements and/or replaces the bar codes 110, 210 of the previous figures. Preferably, the RFID technology should selectively operate over a relatively short range, such as up to 10 cm and/or the like, particularly as needed and/or desired for a particular application. Preferably, the RFID tag 310 is located on a part of the cuff 300 and is overlapped when wrapped around the patient's limb. However, the overlapping part of the cuff 340 is lined with a material that blocks the action of the RFID reader, except for the window 320, through which the RFID tag 310 can be read when the cuff 300 has been wrapped correctly. For example, the cuff 300 can be infused with a layer of radio frequency blocking material 350, and the window 320 provides a break in the radio frequency blocking material through which the RFID tag 310 can be read when the cuff 300 is properly affixed to a particular patient. Preferably, an RFID interrogator (see FIG. 5) associated with the NIBP monitor can read the RFID tag 310 through the window 320, and once the RFID tag 310 is read, then the NIBP monitor can be triggered for initializing the NIBP determinations. And again, the cuff 300 can be further connected to the blood pressure monitor and/or system through a hose 330 and/or the like.

In this embodiment, the cuff size obtained by reading the RFID tag 310 may again be compared with pre-determined cuff size information for ensuring the usage of the correct size of cuff 300. For example, if the cuff size interpreted from the RFID tag 310 does not agree with the pre-determined information for a particular patient, then the processor (see FIG. 5) may again suggest a correct cuff size and/or provide wrapping instructions to the caretaker. The pre-determined information may be obtained, for example, from a server (not shown) having access to the patient's electronic medical record and/or the like, and/or it may be input to the system by the caregiver.

In an embodiment, the cuff size identified using the RFID tag 310 can also be sent to a medical record database (not shown) for storing the same, and the medical record database may be accessed using a server by the NIBP monitoring system.

Referring now to FIG. 4, another blood pressure cuff 400 is provided with a plurality of RFID tags 410 carried on a surface 411 of the cuff 400 for representing the parameter associated with the cuff 400. For example, the plurality of RFID tags 410 may include a first RFID tag 412, a second RFID tag 414, and/or a third RFID tag 416. Preferably, the plurality of RFID tags 410 are provided in association with the cuff 400 for identifying the physical and/or operational characteristics of the cuff 400. Again, the cuff 400 is further provided with an aperture, such as a window 420, for identifying the parameter upon usage of an appropriately-sized cuff and/or proper wrapping thereof. The window 420 provides a visual opening to assist reading the plurality of RFID tags 410 when the cuff 400 is properly sized and applied to the patient's limb, for example using a RFID tag reader (not shown). Preferably, the plurality of RFID tags 410 are printed in a location such that they are overlapped when the cuff 400 is wrapped around the patient's limb, but become detectable through the window 420 when the cuff 400 is properly wrapped around the patient's limb. For example, the degree of wrap of the cuff 400 determines which of the plurality of RFID tags 410 is detectable through the window 420. Thus, the NIBP monitor knows how tightly the cuff 410 is wrapped around the limb of the patient, and it can use this information to more precisely verify proper cuff application and/or adjust its operation. For example, in the figure, the plurality of RFID tags 410 includes the first RFID tag 412, second RFID tag 414, and third RFID tag 416, whereby if the first RFID tag 412 is detected through the window 420, then the cuff 400 may be over-wrapped and the NIBP system may instruct the operator to re-adjust the cuff 400 and/or replace it with one of a different size. Similarly, if the cuff 400 is under-wrapped, then the third RFID tag 216 may be visible through the window 420 and the NIBP system may again instruct the operator to re-adjust the cuff 400 and/or replace it with one of a different size. And likewise, if the cuff 400 is properly sized and wrapped, then the second RFID tag 414 may be detectable through the window 420 and the NIBP system may begin the blood pressure determination and/or allow the clinician to do so. Once the appropriate-sized cuff is wrapped appropriately around the patient's limb, then the correct RFID tag of the plurality of RFID tags 410 will be detectable. And again, the cuff 400 can be further connected to the blood pressure monitor and/or system through a hose 430 and/or the like.

Referring now to FIG. 5, it provides a block diagram 500 of a NIBP measurement system, as described in various embodiments of the inventive arrangements. More specifically, the NIBP system includes a blood pressure cuff 510 and a monitor 520 connected through a connector 530. The cuff 510 is associated with a parameter encoded into an identifier 512, which includes the information about the physical and/or operational characteristics of the cuff 510. The cuff 510 is also provided with an aperture 514 for detecting the identifier 512 upon proper wrapping of the cuff 510 using a correctly-sized cuff. In one embodiment, the aperture 514 preferably comprises a window, provided on and/or carried by a surface of the cuff 510, so that the identifier 512, preferably comprising a barcode, or a plurality thereof, is visible through the aperture 514 upon proper usage of an appropriately-sized cuff 510 and/or proper wrapping thereof. In another embodiment, the aperture 514 preferably comprises a break or interruption, provided on a surface of the cuff 510, so that the identifier 512, preferably comprising a RFID tag, or a plurality thereof, is detectable through the aperture 514 upon proper usage of an appropriately-sized cuff 510 and/or proper wrapping thereof. In this embodiment, the aperture preferably comprises a break or interruption 514 from a layer of radio frequency blocking material within the cuff 510, through which the identifier 512 is detectable through the aperture 514 upon proper usage of an appropriately-sized cuff 510 and/or proper wrapping thereof. The cuff 510, cuff parameters, and aperture 514 construction and/or operation are described in reference to FIGS. 1-4 above. For example, the cuff 510 is preferably connected to the monitor 520 by a wired or wireless connector 530, which acts as an interface between the cuff 510 and monitor 520.

Preferably, the monitor 520 also includes a pressure transducer 522, interrogator 524, processor 526, and/or inflate-deflate mechanism 528. For example, inflating and deflating the cuff 510 can be accomplished using the inflate-deflate mechanism 528, which could include valves, as in conventional NIBP systems.

Preferably, the pressure transducer 522 provides pressure oscillation information to the processor 526 for determining the patient's blood pressure, and the inflate-deflate mechanism 528 can be controlled by the processor 526. Preferably, the interrogator 524 is provided for reading the identifier 512 associated with the cuff 510. In one embodiment, for example, the interrogator 524 includes a barcode reader for reading a barcode associated with the cuff 510. In another embodiment, for example, the interrogator 524 includes a RFID reader for reading a RFID tag associated with the cuff 510. In addition, the interrogator 524 may be operably coupled to the processor 526 for communicating the identified parameter.

Now then, to measure a patient's blood pressure, a clinician and/or the like wraps the cuff 510 around the patient's limb. If the appropriately-sized cuff is used and the wrapping technique is proper, the interrogator 524 identifies the identifier 512 through the aperture 514. If the identifier is read correctly, then the processor 526 may generate and send a signal to the inflate-deflate mechanism 528 to initiate the blood pressure determination. Preferably, the processor 526 is associated with a display (not shown) and based on information obtained from the identifier, instructions to the operator and/or the like may be generated and/or displayed and/or the like.

In one embodiment, the processor 526 compares the cuff size interpreted from the identifier 512 with a pre-determined cuff parameter. The pre-determined cuff parameter may be provided by a caregiver and/or the like and/or the processor 526 may retrieve this information from the patient's medical record, which may be located in a local or remote server and/or the like. If the cuff size interpreted from the identifier 512 and the pre-determined information do not agree, then the processor 526 may suggest the correct cuff size to the caretaker and/or provide wrapping instructions to the caretaker. Preferably, this may be displayed in a display (not shown) associated with the processor 526.

In an embodiment, the barcode and/or RFID tag conveys the cuff size to the NIBP system, which can adjust its operation based on the cuff size, for example, by switching between adult and neonatal modes. Preferably, the NIBP system may then initiate a NIBP determination without further action from the clinician.

In an embodiment, the parameter associated with the cuff 510 may be configured to identify physical attributes and/or operational characteristics of the cuff 510. For example, a cuff's 510 unique serial number may be embedded into the barcode and/or RFID tag for tracking the correct usage of the cuff 510 by the NIBP instruments. This can help ensure that clinicians do not inadvertently exceed the maximum rated life of a cuff 510, for example.

In an embodiment, the identifier 512 can be provided with one or more bar codes. The visibility of such barcodes through the aperture 514 can indicate a degree of wrap of the cuff 510. Based on the read barcode, the system may generate wrapping instructions and/or provide additional information to an operator. Preferably, the NIBP system may also adjust its operation based on the cuff size and/or degree of wrap.

In another embodiment, the identifier 512 can be provided with one or more RFID tags. The detectability of such RFID tags through the aperture 514 can indicate a degree of wrap of the cuff 510. Based on the read barcode, the system may generate wrapping instructions and/or provide additional information to an operator. Preferably, the NIBP system may also adjust its operation based on the cuff size and/or degree of wrap.

In the same or different embodiments, a cuff's 510 unique serial number can also be combined with an inventory control system, for example, to facilitate ordering additional cuffs and/or supplies as they are depleted.

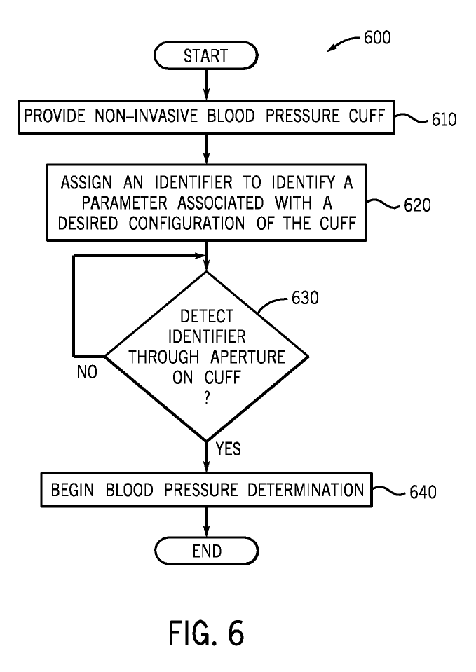

Referring now to FIG. 6, it provides a flowchart 600 for illustrating various methods of controlling blood pressure determination as described in various embodiments of the inventive arrangements. More specifically, at a step 610, a NIBP cuff is provided to a patient, which is generally worn about the limb of a patient. Preferably, the cuff is selectively deflatable and inflatable and used in monitoring the blood pressure using an NIBP monitoring session. At a step 620, an identifier is assigned to identify a parameter associated with the cuff, such as the size of the cuff. Preferably, the identifier is provided on a surface of the cuff. Preferably, the identifier could be one or more bar codes and/or RFID tags. At a step 630, if the identifier is viewed and/or otherwise detected through an aperture provided on or supported by the cuff, then the blood pressure determination can begin in a step 640, after which the method ends. Preferably, the identifier and aperture are located on the cuff such that the identifier is visible through the aperture when the cuff is sized appropriately and/or worn properly. Otherwise, control remains at step 630 until the identifier is viewed or otherwise detected. At step 640, the operation of the NIBP monitor is controlled using the detected identifier. For example, if the identifier is detected correctly at step 630, then the operation of the NIBP monitor can be triggered at step 640. Otherwise, a processor and/or the like may be configured to give proper wrapping instructions to the operator and/or the like and/or suggest an appropriate cuff size.

Accordingly, the inventive arrangements can improve the robustness of NIBP systems. They can reduce the chances that inaccurate NIBP measurements are taken, thereby reducing the likelihood of medical errors based on this information.

The inventive arrangements can also enhance the trustworthiness of NIBP determinations, particularly when taken by lesser-skilled operators. This can enhance a healthcare organization's ability to leverage less-expensive labor, such as technicians and/or assistants, instead of registered nurses, for determinations such as a patient's blood pressure.

In addition, automated triggering mechanism can improve the ease-of-use of many NIBP instruments, particularly when they are wall-mounted and/or the like.

Thus, various embodiments of NIBP monitoring systems are disclosed, and the inventive arrangements describes various systems and methods that facilitate using an appropriate-sized cuff in NIBP monitoring.

While the inventive arrangements have been described with reference to preferred embodiments, those skilled in the art will appreciate that certain substitutions, alterations and omissions may be made to the embodiments without departing from the spirit of the inventive arrangements. Accordingly, the foregoing description is meant to be exemplary only, and should not limit the scope of the inventive arrangements as set forth in the following claims.

1. A non-invasive blood pressure system, comprising:
a blood pressure cuff having an identifier and a non-insertion aperture such that said identifier can be read through said aperture when said cuff is in a desired configuration.

2. The system of claim 1, wherein said identifier comprises one or more bar codes.

3. The system of claim 1, wherein said identifier comprises one or more radio-frequency identification tags.

4. The system of claim 3, wherein said cuff comprises radio-frequency blocking material except about said aperture.

5. The system of claim 1, wherein said identifier can be read through said aperture only when said cuff is in said desired configuration.

6. The system of claim 1, wherein said desired configuration comprises a desired degree of wrap of said cuff.

7. The system of claim 1, wherein said desired configuration comprises a desired sizing of said cuff for a particular patient.

8. The system of claim 1, wherein said is a visual aperture.

9. The system of claim 1, wherein said aperture is a line-of-sight aperture.

10. The system of claim 1, wherein at least one of said identifier and said aperture are provided on a surface of said cuff.

11. A non-invasive blood pressure system, comprising:
a blood pressure cuff having an identifier a non-insertion aperture such that said identifier can be read through said aperture when said cuff is in a desired configuration; and
a processor in operative communication with said cuff and configured to initiate a blood pressure determination when said identifier is read through said aperture.

12. The system of claim 11, wherein said identifier comprises one or more bar codes.

13. The system of claim 11, wherein said identifier comprises one or more radio-frequency identification tags.

14. The system of claim 13, wherein said cuff comprises radio-frequency blocking material except about said aperture.

15. The system of claim 11, wherein said identifier can be read through said aperture only when said cuff is in said desired configuration.

16. The system of claim 11, wherein said processor is configured to automatically initiate said determination when said identifier is read through said aperture.

17. A method of determining blood pressure, comprising:
providing a blood pressure cuff having an identifier and a non-insertion aperture such that said identifier can be read through said aperture when said cuff is in a desired configuration.

18. The method of claim 17, wherein said identifier comprises one or more bar codes.

19. The method of claim 17, wherein said identifier comprises one or more radio-frequency identification tags.

20. A method of determining blood pressure, comprising:
providing a blood pressure cuff having an identifier and a non-insertion aperture such that said identifier can be read through said aperture when said cuff is in a desired configuration: and
initiating a blood pressure determination only when said identifier is read through said aperture.
